(19) United States
(12) Patent Application Publication
(10) Pub. No.: US 2025/0230397 A1
(43) Pub. Date: Jul. 17, 2025

(54) SYSTEMS AND METHODS INVOLVING CONTROLLED OXYGEN RELEASE IN BIOMATERIALS

(71) Applicant: Sartorius Stedim Biotech GmbH, Göttingen (DE)

(72) Inventors: Samin Akbari, Winchester, MA (US); Oscar Reif, Hannover (DE)

(73) Assignee: Sartorius Stedim Biotech GmbH, Göttingen (DE)

(21) Appl. No.: 18/853,505

(22) PCT Filed: Mar. 29, 2023
(86) PCT No.: PCT/IB2023/000185

§ 371 Date: Oct. 2, 2024

Related U.S. Application Data
(60) Provisional application No. 63/327,260, filed on Apr. 4, 2022.

(51) Int. Cl. C12M 1/34; C12M 1/00; C12M 3/06

(52) U.S. Cl. CPC C12M 41/34; C12M 23/14; C12M 23/16; C12M 23/40; C12M 29/06; C12M 41/12

(57) ABSTRACT

Some aspects of the disclosure are related to systems and methods for controlled oxygen release from biomaterials in vessels and unit operations or components of cell culture, cell containment, and/or bioreactor. Vessels, unit operations, devices, and/or components of the invention may be used to perform all or part of a biological and/or chemical process involving biologicals (e.g., a plurality of cells) in the presence of oxygen-releasing agents. In some embodiments, a system comprises a vessel comprises an oxygen-releasing agent configured to generate in-situ and release oxygen in a sustained manner. The presence of an oxygen-releasing agent may advantageously allow for high cell density fermentation and cell cultivation in a vessel and provide an alternative for supplemental gassing means (e.g., sparger, etc.). Some embodiments of the disclosure are directed to employing the oxygen-releasing agents in microfluidic or millifluidic systems.

### CROSS-REFERENCE TO RELATED APPLICATIONS

This application claims the benefit of priority under 35 U.S.C. § 119(e) of U.S. Provisional Application No. 63/327,260, filed Apr. 4, 2022, the disclosure of which is incorporated by reference herein in its entirety.

### TECHNICAL FIELD

The present invention relates generally to biomaterials, and more specifically, to systems and methods involving controlled oxygen release in biomaterials.

### BACKGROUND

A variety of vessels for manipulating fluids and/or for carrying out biological and/or chemical reactions are available. Traditional bioreactors, which are typically designed as stationary vessels housing a plurality of cells, typically lack efficient means for providing an adequate supply of dissolved oxygen (i.e., dissolved O₂) in the cell media to the cells in a controlled manner. For example, in conventionally aerated bioreactors, low oxygen solubility and slow oxygen transfer rates often lead to slow cell growth in addition to other negative effects on the cells. Although gassing systems are known, improvements and/or alternatives to such systems would be beneficial.

### SUMMARY

The present invention relates generally to biomaterials, and more specifically, to systems and methods involving controlled oxygen release in biomaterials. The subject matter of the present invention involves, in some cases, interrelated products, alternative solutions to a particular problem, and/or a plurality of different uses of one or more systems and/or articles.

In some aspects, systems for bioprocessing are provided.

In some embodiments, a system for bioprocessing comprises a vessel comprising an inlet configured to introduce substances into the vessel, wherein the vessel comprises at least one microfluidic or millifluidic channel; a plurality of cells contained within the vessel; and an oxygen-releasing agent disposed within the vessel.

In some embodiments, a system for bioprocessing comprises a vessel comprising an inlet configured to introduce substances into the vessel; a plurality of cells suspended in a fluid or attached to a carrier contained within the vessel; and an oxygen-releasing agent disposed within the vessel, wherein the oxygen-releasing agent is incapable of reversibly solubilizing and/or binding oxygen.

In some aspects, a series of methods are provided.

In some embodiments, a method comprises providing a system comprising a vessel, wherein the vessel comprises an inlet and at least one microfluidic or millifluidic channel; disposing an oxygen-releasing agent into the vessel; introducing at least one cell into the vessel; and releasing oxygen in the vessel from the oxygen-releasing agent.

In some embodiments, the method comprises providing a system comprising a vessel, wherein the vessel comprises an inlet; disposing an oxygen-releasing agent into the vessel, wherein the oxygen-releasing agent is incapable of reversibly solubilizing and/or binding oxygen; introducing at least one cell suspended in a fluid or attached to a carrier into the vessel; and releasing oxygen in the vessel from the oxygen-releasing agent.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a cross-sectional view of a first system for bioprocessing, according to some embodiments;

FIG. 2 is a cross-sectional view of a second system for bioprocessing, according to some embodiments;

FIG. 3 is a side view of a microfluidic or millifluidic channel, according to some embodiments;

FIG. 4A is a schematic depiction of a microcarrier or nanocarrier comprising an oxygen-releasing agent, according to some embodiments;

FIG. 4B is a schematic depiction of a film or slab comprising an oxygen-releasing agent, according to some embodiments;

FIG. 4C is a schematic depiction of a coating comprising an oxygen-releasing agent, according to some embodiments;

FIG. 4D is a schematic depiction of a hydrogel scaffold comprising an oxygen-releasing agent, according to some embodiments;

FIG. 5 is a schematic depiction of a microcarrier containing at least one cell, according to some embodiments; and

FIG. 6 is a flow diagram of a method for releasing oxygen in a system for bioprocessing, according to some embodiments.

### DETAILED DESCRIPTION

The present disclosure relates in some aspects to systems and methods for controlled oxygen release from biomaterials. In some embodiments, the present disclosure is directed to controlling oxygen release in vessels and unit operations or components of cell culture, cell containment, and/or bioreactor. The vessels, unit operations, devices, and/or components disclosed herein may be used to perform all or part of a biological and/or chemical process involving biologicals (e.g., a plurality of cells) in the presence of oxygen-releasing agents. For example, a system may comprise a vessel containing a plurality of cells and an oxygen-releasing agent configured to release and supply oxygen to the cells in a controlled and sustained manner.

The presence of an oxygen-releasing agent may advantageously allow for high cell density fermentation and cell cultivation in a vessel. For example, in some existing systems for bioprocessing, the supply of oxygen to the cells therein may be limited by the solubility limit of oxygen in the fluid (e.g., cell media) adjacent the cells. The use of oxygen-releasing agent may provide an additional source of oxygen that can be released in-situ and increase the amount of dissolved oxygen readily available for use by the cells. Moreover, the oxygen-releasing agents may serve as an alternative for conventional gassing means (e.g., sparger, etc.). Such an alternative oxygen supply may be particularly advantageous for systems having small sizes and volumes, e.g., microfluidic or millifluidic systems, where physical limitations associated with these systems may render the installation of conventional gassing means challenging. As used herein, a microfluidic system refers to a device, apparatus or system including at least one fluid conduit or channel (i.e., a microfluidic channel) having a cross-sectional dimension of less than about 1 millimeter (mm). Similarly, a millifluidic system refers to a device, apparatus or system including at least one fluid conduit or channel (i.e., a millifluidic channel) having a cross-sectional dimension of less than about 1 centimeter (cm).

Although much of the description herein involves an exemplary application of the present disclosure related to bioreactors (and/or biochemical and chemical reaction systems), the disclosure and its uses are not so limited, and it should be understood that aspects of the invention can also be used in other settings, including those involving containment systems in general, as well as systems for containment and/or processing of a fluid in a vessel (e.g., mixing systems). Other applications may include chemical manufacturing systems, pharmaceutical manufacturing systems, etc.

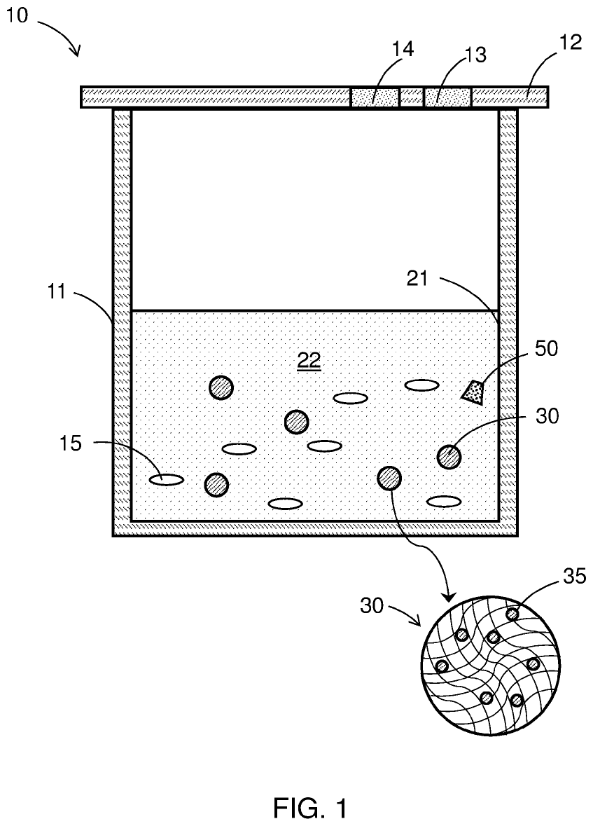

In some embodiments, a system for bioprocessing is provided. In some embodiments, the system is a bioreactor and/or a biochemical and chemical reaction system. In one set of embodiments, the system comprises a vessel. In some embodiments, the vessel is a reaction vessel. The vessel may include one or more openings (e.g., an inlet, an outlet, etc.) configured to introduce or remove substances (e.g., cell media, cells) into the vessel. A non-limiting representation of one such embodiment is shown in FIG. 1. As shown, a system 10 for bioprocessing comprises a vessel 11 that includes a first opening 13 (e.g., an inlet) configured to introduce substances into the vessel 11 and a second opening 14 (e.g., an outlet) configured to remove substances from the vessel 11. The various openings may allow for material exchange within the vessel, e.g., exchange of various components (e.g., cell media, oxygen-releasing agents, ancillary agents, etc.) and/or harvesting of cells.

In some embodiments, the system comprises a plurality of cells contained within the vessel. In one set of embodiments, the plurality of cells may be suspended (e.g., dispersed) in a fluid within the vessel. For example, as shown in FIG. 1, the system 10 comprises a plurality of cells 15 suspended in a fluid 22 within the vessel 11. In some cases, a substantial percentage (e.g., at least 50%, at least 60%, at least 70%, at least 80%, at least 85%, at least 90%, at least 95%, at least 97%, at least 99%, or all) of the plurality of cells may be suspended (e.g., dispersed) in the fluid within the vessel without forming an interconnected network of cells (e.g., a tissue). That is, the suspended cells may be dispersed individually (e.g., not connected to one another) such that an interconnected network of cells is not formed, or is minimally-formed. For example, as shown, FIG. 1 illustrates one embodiment in which each of the plurality of cells 15 is suspended (e.g., dispersed) in the fluid 22 without being connected to one another or forming an interconnected network of cells.

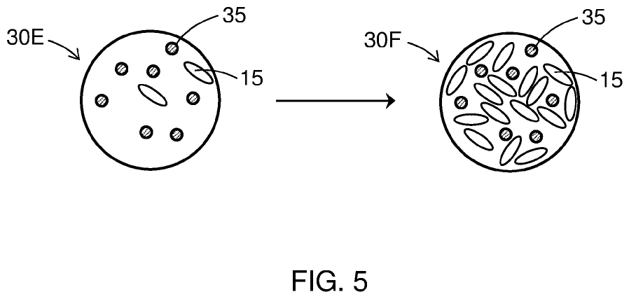

Alternatively or additionally, in some embodiments, at least some of the plurality of cells may be attached to a carrier (e.g., microparticles, microcarriers, etc.) suspended within a fluid within the vessel. For example, in FIG. 1, at least some of a plurality of cells (not shown) may be attached to the carrier 30. As described in more detail below, FIG. 5 illustrates a non-limiting representation of a carrier 30F containing oxygen-releasing agent 35 and a plurality of cells 15 attached to the surface of the carrier 30F. In some embodiments, the carrier (e.g., a microcarrier) may be advantageously employed as a platform for cell culturing.

In some cases, the vessel may form a closed system. A closed system may be a system that is not in fluidic communication with the external environment. For example, as shown in FIG. 1, a lid 12 may be disposed on the vessel 11 and thus forms a closed system. In some cases, a biological reaction and/or biological processing may be carried out within the closed system. For example, the vessel may be a closed system configured to carry out a biological reaction and/or biological processing (e.g., cell growth, cell culturing, etc.) via the plurality of cells.

In some embodiments, the plurality of cells may be contained within the vessel in the presence of an oxygen-releasing agent. An oxygen-releasing agent, according to some embodiments, may be a material and/or a compound that is configured to release or generate oxygen in-situ to its surrounding environment. For example, the oxygen-releasing agent may be configured to release or generate oxygen, and supply oxygen to the plurality of cells in the vessel. As discussed in more detail below, any of a variety of appropriate oxygen-releasing agents may be employed in the systems described herein.

The oxygen-releasing agent may be disposed within the vessel in any of a variety of appropriate configurations. In one set of embodiments, the oxygen-releasing agent may be contained (e.g., encapsulated, entrapped, localized, etc.) within a carrier (e.g., a microcarrier). As shown illustratively in FIG. 1, an oxygen-releasing agent 35 may be contained with a carrier 30 that is disposed within (e.g., suspended) the vessel 20. The carrier may have any of a variety of shapes, configurations, and/or dimensions. Non-limiting examples of a carrier include one or more of particles (e.g., microparticle, microcapsule, nanoparticle), a droplet (e.g., emulsion droplet), a film, a slab, a pill, a collapsible bag (e.g., a tea bag, a dialysis bag), etc.

While FIG. 1 shows a set of embodiments in which the oxygen-releasing agent is disposed within the vessel via a carrier, it should be understood that not all embodiments described herein are so limiting, and that in other embodiments, the oxygen-releasing agent may be disposed directly into the vessel without the use of a carrier, as described in more detail below.

In some embodiments, the system for bioprocessing (e.g., a bioreactor) described herein is a microfluidic system or millifluidic system. As described above, a microfluidic system refers to a device, apparatus or system including at least one fluid conduit or channel (i.e., a microfluidic channel) having a cross-sectional dimension of less than about 1 millimeter (mm). Similarly, a millifluidic system refers to a device, apparatus or system including at least one fluid conduit or channel (i.e., a millifluidic channel) having a cross-sectional dimension of less than about 1 centimeter (cm). In some instances, a microfluidic or millifluidic system may include a vessel comprising a microfluidic or millifluidic conduit or channel, respectively. In some instances, the vessel itself may be a microfluidic or millifluidic conduit or channel. For example, as shown in FIG. 1, the system 10 may be either a microfluidic or millifluidic system comprising the vessel 11. The vessel 11 may have a dimension and volume representative of a microfluidic or millifluidic conduit or channel. In cross-section, the conduit or channel may be rectangular or substantially non-rectangular, such as circular or elliptical. In some embodiments, the conduit or channel may be a capillary tube.

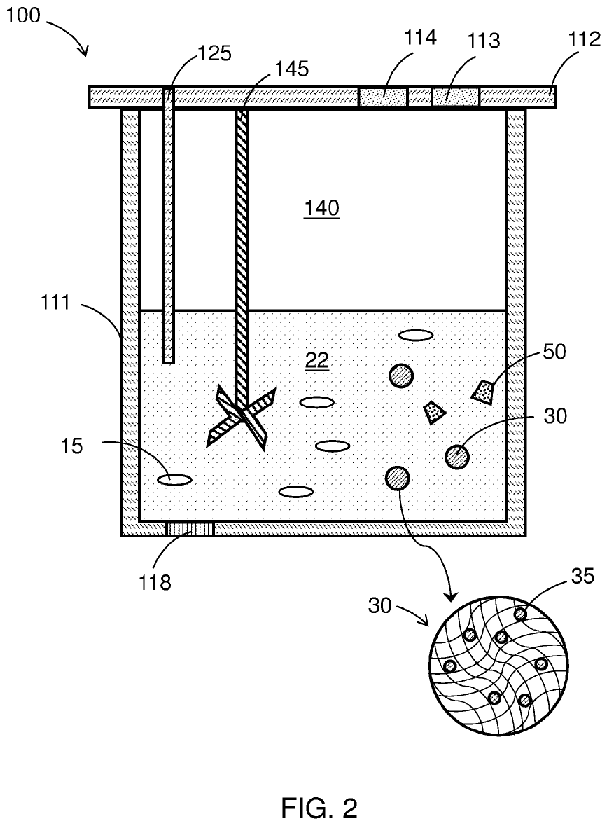

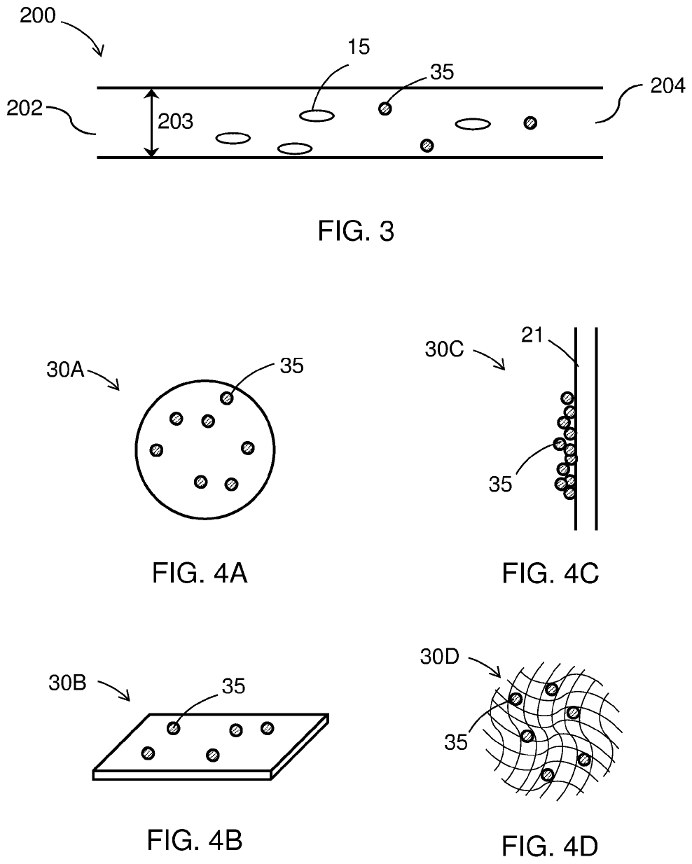

A non-limiting embodiments of a microfluidic or millifluidic channel is illustrated in FIG. 3. As shown in FIG. 3, a microfluidic channel 200 may comprise an inlet 202 configured to introduce substances (e.g., at least one cells, cell media, oxygen-releasing agents, etc.) into the channel and an outlet 204 configured to remove substances from the channel. In some embodiments, at least one or more cells in a fluid are contained within the microfluidic or millifluidic channel. Additionally, one or more oxygen-releasing agents may be dispersed in the microfluidic or millifluidic channel in any appropriate form, e.g., with or without the use of a carrier described elsewhere herein. For example, in FIG. 3, at least one or more cells suspended in a fluid may be contained within the channel 200. Additionally, one or more oxygen-releasing agent 35 may be dispersed in the channel 200. In some instances, the one or more oxygen-releasing agent 35 may be dispersed in a carrier, e.g., such as a microparticle or nanoparticle (e.g., similar to carrier 30 in FIG. 2), a slab, a film, a coating, a hydrogel scaffold, etc. The microfluidic or millifluidic channel 20 may have a cross-sectional dimension 203 in one or more of the microfluidic or millifluidic size ranges described below. In some embodiments, the at least one or more cells may be attached to a carrier described elsewhere herein.

In some embodiments, the vessel may comprise a conduit or channel in the microfluidic size range and may have, for example, an average cross-sectional dimension (e.g., width, height), or portions having a cross-sectional dimension, of no more than 1 millimeter, no more than 500 micrometers, no more than 100 micrometers, no more than 50 micrometers, no more than 10 micrometers, no more than 5 micrometers, or no more than 1 micrometer. In some embodiments, the vessel may comprise a conduit or channel in the microfluidic size range and may have, for example, average cross-sectional dimension (e.g., width, height), or portions having a cross-sectional dimension, of at least 0.1 micrometers, at least 1 micrometer, at least 5 micrometers, at least 10 micrometers, at least 50 micrometers, at least 100 micrometers, or at least 500 micrometers. Combination of the above-reference ranges are possible (e.g., between 0.1 micrometers and 1 millimeter). Other ranges are also possible.

In some embodiments, the vessel may comprise a conduit or channel in the millifluidic size range and may have, for example, average cross-sectional dimension (e.g., width, height), or portions having a cross-sectional dimension, of no more than 10 millimeters, no more than 5 millimeters, or no more than 2 millimeters. In some embodiments, the vessel may comprise a conduit or channel in the millifluidic size range and may have, for example, average cross-sectional dimension, or portions having a cross-sectional dimension, of at least 1 millimeter, at least 2 millimeters, or at least 5 millimeters. Combination of the above-reference ranges are possible (e.g., between 1 millimeter and 10 millimeters). Other ranges are also possible.

A system (e.g., a millifluidic or microfluidic system) may comprise a vessel having any of a variety of appropriate volumes described herein. In some embodiments, the vessel may have a volume of greater than or equal to 1 μL, greater than or equal to 5 μL, greater than or equal to 10 μL, greater than or equal to 50 μL, greater than or equal to 100 μL, greater than or equal to 500 μL, greater than or equal to 1 mL, greater than or equal to 2 mL, greater than or equal to 4 mL, greater than or equal to 6 mL, or greater than or equal to 8 mL. In some embodiments, the vessel may have a volume of less than or equal to 10 mL, less than or equal to 8 mL, less than or equal to 6 mL, less than or equal to 4 mL, less than or equal to 2 mL, less than or equal to 1 mL, less than or equal to 500 μL, less than or equal to 100 μL, less than or equal to 50 μL, less than or equal to 10 μL, or less than or equal to 5 μL. Combination of the above-reference ranges are possible (e.g., greater than or equal to 1 μL and less than or equal to 4 mL). Other ranges are also possible.

A microfluidic or millifluidic system may have any suitable components and properties (e.g., an inlet, a plurality of cells suspended in a fluid within the vessel, an oxygen-releasing agent, etc.) described herein with respect to the system in FIG. 1. In some embodiments, the presence of an oxygen-releasing agent disposed within a vessel may advantageously provide sufficient oxygen to support and promote cell viability within the vessel. The use of oxygen-releasing agent may be particularly advantageous for such systems having small sizes and volumes, where it may be physically challenging to incorporate gassing means (e.g., tubes, lines, sparges).

In some embodiments, the system described herein may include various additional components, e.g., mixing mechanisms, sensors, supplemental gassing lines, etc. A non-limiting example of one such embodiment is shown in FIG. 2. As shown illustratively, the system may comprise any of the components described herein with respect to FIG. 1, including but not limited to, a plurality of cells suspended in a fluid or attached to a carrier contained within the vessel, an oxygen-releasing agent disposed within the vessel, etc. For example, a system 100 may comprise a vessel 111, the plurality of cells 15 suspended in the fluid 22 contained within the vessel 111, and the oxygen-releasing agent 35 disposed within the carrier 30. The oxygen-releasing agent may be disposed within the vessel in any of a variety of appropriate forms and configurations described herein with respect to FIG. 1.

In some embodiments, the vessel forms a closed system. As shown in FIG. 2, a vessel lid 112 may be disposed at the opening of the vessel, thereby forming a closed system. In some embodiments, the vessel may comprise one or more openings (e.g., an inlet, an outlet, etc.) that can be used to introduce and/or remove substances into or from the vessel. For example, as shown in FIG. 2, the vessel 111 comprises a first opening 113 (e.g., an inlet) and a second opening 114 (e.g., an outlet) positioned on the vessel lid 112 configured to introduce and/or remove substances, e.g., the plurality of cells 15, the fluid 22, and/or the oxygen-releasing agent 35, into the vessel 111.

In some embodiments, the system may optionally comprise a conduit (e.g., a sparger) in fluid communication with the vessel configured to introduce a gas into the vessel. For example, in one set of embodiments, in addition to containing oxygen-releasing agents, the vessel may also be connected via one or more conduits to one or more sources of gases such as air, oxygen, carbon dioxide, nitrogen, ammonia, or mixtures thereof, in various aspects of the disclosure. In some embodiments, systems containing a conduit for gassing means may have dimensions larger than microfluidic or millifluidic systems. As shown in FIG. 2, the system 100 may comprise a conduit 125 that is in fluid communication with the inside of the vessel 111 and may be configured to introduce a gas into the vessel 111. A source of gas may be connected to the conduit. The gas may comprise any appropriate types of gas (e.g., oxygen gas, nitrogen gas, and/or carbon dioxide gas, etc.) that can promote the viability of cells disposed within the vessel and/or facilitates a biological process within the vessel.

In some embodiments, the conduit may be in fluidic communication with a fluid within the vessel, such that the gas may be delivered to the plurality of cells contained within the vessel. In some such embodiments, at least a portion of the delivered gas may be dissolved by the fluid. For example, as shown in FIG. 2, the conduit 125 may extend into the fluid 22 such that a gas may be delivered through the conduit 125 to the plurality of cells 15 suspended in the fluid 22.

While FIG. 2 shows a set of embodiments in which a conduit in configured to be in fluid communication with the fluid comprising a plurality of cells, it should be understood that not all embodiments described herein are so limiting, and in other embodiments, the conduit may not be in fluidic communication with the fluid comprising the plurality of cells. For example, alternatively, the conduit may extend into a headspace (e.g., a headspace 140) of the vessel such that a gas is delivered into the headspace of the vessel. In some instances, at least a portion of the gas within the headspace may be subsequently dissolved by the fluid in which the plurality of cells is suspended in.

The systems described herein may optionally further comprise various additional components, as described in more detail below, e.g., such as mixing mechanisms, sensors, pump, ancillary agents, heating mechanisms, etc.

In some embodiments, the system comprises a mixing mechanism. For example, as shown in FIG. 2, the vessel 111 may further comprise a mixing mechanism 145 disposed within the vessel 111. The mixing mechanism may be configured to facilitate efficient mixing of substances (e.g., fluid 22, plurality of cells 15, carrier 30, oxygen-releasing agent 35, dissolved gases, etc.) disposed within the vessel. For instance, the mixing mechanism may advantageously allow for efficient mixing of dissolved gases (released by the oxygen-releasing agent 35 and/or delivered from a source via conduit 125) within the fluid 22. An efficient mixing may ensure uniform exposure of dissolved gases to and uptake of dissolved gases by the plurality of cells contained within the vessel.

In some embodiments, the system may comprise a sensor configured to measure a property within the vessel. For example, as shown in FIG. 2, the system 100 comprises a sensor 118 positioned within the vessel 111 configured to measure a property within the vessel 111. The property may be a property that is associated with the substances (e.g., the fluid, the cells disposed within the fluid, oxygen-releasing agents, etc.) residing within the vessel. In one set of embodiments, the measured property comprises a dissolved oxygen (DO) level, a pressure, a temperature and/or pH of the fluid within the vessel. In some such embodiments, the sensor comprises one or more of a luminescent DO sensor, a pH sensor, a temperature sensor, and/or a pressure sensor.

While FIG. 2 shows a set of embodiments in which the sensor is positioned at the base of the vessel, it should be understood that not all embodiments described herein are so limiting, and that in some embodiments, the sensor may be positioned in any of a variety of locations in the vessel.

In some embodiments, the system optionally comprises an ancillary agent having an affinity for a gaseous byproduct (e.g., CO₂) produced from a bioprocessing and/or biochemical process within the vessel. For example, as shown in FIGS. 1-2, an ancillary agent 50 may be disposed in the fluid 22 within the vessel 11 or 111. As described in more detail below, the ancillary agent may include any of a variety of compounds, materials, and/or devices capable of associating (e.g., binding to, interacting with, etc.) with a gaseous product.

As noted above, in some instances, the system for bioprocessing may be a bioreactor. Any of a variety of suitable bioreactors may be employed for bioprocessing. Non-limiting examples of bioreactors include Sartorius bioreactors and other bioreactors, e.g., such as Biostat STR® Bioreactors, Biostat® RM and/or RM perfusion Bioreactors, Ambr® Bioreactors, Mobius® Bioreactors, etc.

Some aspects of the disclosure are directed to methods of oxygen release or generation in a system (e.g., systems 10,100, and 200 in FIGS. 1-3) for carrying out bioprocessing and/or biochemical reactions.

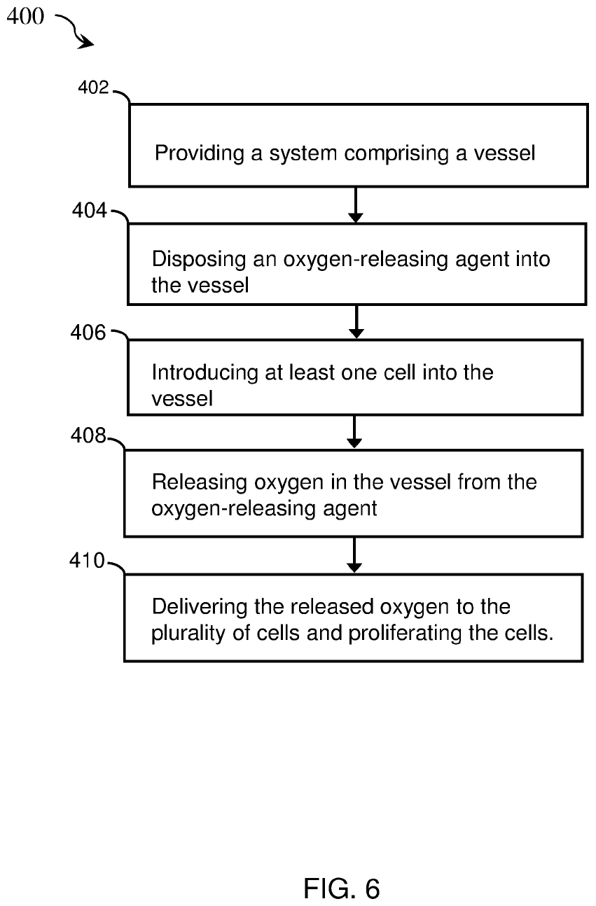

In some embodiments, the method comprises providing a system comprising a vessel, e.g., as shown in step 402 in FIG. 6. The system and/or vessel may comprise any of a variety of components (e.g., inlet, outlet, vessel lid, sensors, etc.) and materials (e.g., carrier, oxygen-releasing agent, ancillary agent, plurality of cells suspended in a fluid, etc.) described herein with respect to FIGS. 1-2. In some embodiments, the system is a microfluidic or millifluidic system as described above (e.g., systems 10 and 200 in FIGS. 1 and 3). For example, the microfluidic or millifluidic system may comprise (or itself may be) at least one microfluidic or millifluidic channel having a cross-sectional dimension in one or more ranges described with respect to FIG. 3.

In some embodiments, the method comprises introducing a plurality of substances via an inlet into the vessel. In one set of embodiments, the method comprises disposing an oxygen-releasing agent into the vessel via an opening (e.g., an inlet), e.g., as shown in step 404 of FIG. 6. In addition, according to some embodiments, at least one cell of a biological nature may be introduced into the vessel via the opening (e.g., an inlet), e.g., as shown in step 406. In some cases, the at least one cell may be suspended in a fluid or attached to a carrier contained within the vessel. For example, referring again to FIGS. 1-3, the plurality of substances may be introduced into the vessel 11 or 111 via the opening 13 or 113. The substances may include the plurality of cells 15, the fluid 22, the carrier 30 comprising the oxygen-releasing agent 35, the ancillary agent 50, etc.

While FIGS. 1-2 illustrate a set of embodiments in which the oxygen-releasing agent is contained within a carrier, it should be understood that not all embodiments described herein are so limiting, and that in some embodiments, the oxygen-releasing agent may have any appropriate form described herein and may be introduced into the vessel with or without the use of a carrier. For example, FIG. 3 illustrates an embodiment in which the oxygen-releasing agent is not contained within a carrier.

It should be noted that the various substances described herein may be introduced into the vessel in any appropriate order. For example, in one set of embodiments, the fluid containing the at least one cell may be introduced prior to introducing the oxygen-releasing agents and/or ancillary agents. In another set of embodiments, the oxygen-releasing agents and/or ancillary agents may be introduced into the vessel prior to introducing the fluid containing the at least one cell. In some embodiments, at least two (e.g., at least three, or all) of the substances (e.g., cells, oxygen-releasing agent, ancillary agent, etc.) may be introduced into the vessel at the same time.

In some embodiments, the method comprises releasing oxygen in the vessel from the oxygen-releasing agent, e.g., as shown in step 408 of FIG. 6.

In some embodiments, releasing oxygen comprises generating oxygen via a reaction or unbinding (e.g., dissociating) oxygen from the oxygen-releasing agent. In one set of embodiments, the oxygen-releasing agent may include a material capable of reversibly solubilizing or binding oxygen. In some such embodiments, oxygen may be released to the environment via dissociation of oxygen from the oxygen-releasing agent. Alternatively or additionally, in some embodiments, the oxygen-releasing agent may include a material capable of undergoing a reaction to generate oxygen. In some such cases, oxygen may be generated in-situ and released to the environment upon in-situ generation. As shown in FIGS. 1-3, the oxygen-releasing agent 35 may release the generated or released oxygen to its surrounding environment.

The released oxygen may then be delivered to the plurality of cells and contribute to the proliferation of the cells, e.g., as shown in step 410 of FIG. 6. In some embodiments, the oxygen-releasing agent may allow for oxygen generation or release in a sustained and controlled manner. In some embodiments, the oxygen-releasing agent may generate or release oxygen at a rate that is beneficial for cell proliferation.

As described in more detail below, the method of in-situ oxygen generation may depend on the type of oxygen-releasing agent employed in the system. In one set of embodiments, oxygen may be produced as a reaction product between the oxygen-releasing agent and various substances. Alternatively, the oxygen-releasing agent may have a relatively high capacity for storing (e.g., binding and/or solubilizing) oxygen. In some such cases, releasing oxygen comprises releasing and/or dissociating oxygen from the oxygen-generating agent.

In some embodiments, the method optionally comprises introducing a gas (e.g., O₂, CO₂, and/or N₂) into the vessel via a conduit. As shown in FIG. 2, in system 100, a gas may be introduced via the conduit 125 into the fluid 15 in the vessel 111. The introduced gas may be delivered to the plurality of cells and contribute to the proliferation of the cells.

In some embodiments, the method optionally comprises mixing the various substances within the vessel via a mixing mechanism. For example, as shown in FIG. 2, the mixing mechanism 145 may be employed to facilitate efficient mixing of the various substances (e.g., cells, cell media, various agents) and gases within the vessel 111.

In some embodiments, the method comprises controlling, at least in part, via the oxygen-releasing agent, a level of dissolved oxygen in the vessel when the dissolved oxygen level is below a threshold level. In some such embodiments, the step of controlling oxygen level comprises measuring a level of dissolved oxygen within the vessel via a sensor (e.g., a dissolved oxygen sensor). For example, as shown in FIG. 2, the dissolved oxygen within the fluid 22 may be detected and monitored by the sensor 118. In some embodiments, upon detecting an oxygen level below a threshold level within the vessel, additional oxygen-releasing agent may be introduced into the vessel. Alternatively or additionally, the conduit (e.g., conduit 125) may be triggered to introduce oxygen into the vessel.

In some embodiments, the method comprises controlling a level of dissolved oxygen in the vessel by adjusting an amount and/or type of the oxygen-releasing agent within the vessel. For example, by measuring the level of dissolved oxygen within the fluid containing the plurality of cells, the measured value of dissolved oxygen may be compared to a predetermined threshold value of oxygen necessary for cell growth, proliferation and/or bioprocessing. Accordingly, based on the measured value, a desirable amount and/or type of oxygen-releasing agent may be introduced into the vessel. In some cases, as described in more detail below, the sensor may communicate with a controller associated with the system. The controller may be configured in turn to control the addition of the oxygen-releasing agent into the vessel.

In some embodiments, the presence of oxygen-releasing agents may contribute to a relatively high level of cell proliferation. For example, in some embodiments, cells contained within the fluid in the vessel may be proliferated to an amount of at least 2 times, at least 3 times, at least 5 times, at least 10 times, at least 15 times, at least 20 times, at least 25 times, at least 50 times, at least 75 times, at least 100 times, at least 200 times, at least 400 times, at least 600 times, at least 800 times, at least 1000 times, at least 1500 times, at least 2000 times, at least 3000 times, or at least 4000 times an original number of cells introduced into the vessel. For example, in some embodiments, cells contained within the fluid in the vessels may be proliferated to an amount of no more than 5000 times, no more than 4000 times, no more than no more than 3000 times, no more than no more than 2000 times, no more than no more than 1000 times, no more than 800 times, no more than 600 times, no more than 400 times, no more than 200 times, no more than 100 times, no more than 55 times, no more than 50 times, no more than 25 times, no more than 20 times, no more than 10 times, no more than 5 times, or no more than 3 time an original number of cells introduced into the vessel. Combination of the above-reference ranges are possible (e.g., at least 2 times and no more than 5000 times, or at least 2 times and no more than 1000 times). Other ranges are also possible.

The oxygen-releasing agent may include any of a variety of appropriate materials and/or compounds capable of releasing and/or generating oxygen gas. Non-limiting examples of oxygen-releasing agents include peroxides such as a solid inorganic peroxide, a liquid peroxide, and/or a fluorinated compound. Specific non-limiting examples include calcium peroxide, magnesium peroxide, sodium peroxide; sodium percarbonate, hydrogen peroxide, fluorinated compounds (e.g., perfluorodecalin, perflubron, etc.), etc.

In one set of embodiments, the oxygen-releasing agent may be capable of reversibly solubilizing or binding oxygen. In some cases, the oxygen-releasing agent may have a relatively high oxygen-carrying capacity. Non-limiting examples of such oxygen-releasing agent includes various types of fluorinated compounds such as polyfluorinated and/or perfluorinated compounds (e.g., fluorinated, polyfluorinated, and/or perfluorinated oils). In some cases, the oxygen-releasing agent may be capable of releasing the stored oxygen to its surrounding environment via diffusion.

In some embodiments, the oxygen-releasing agent comprises a fluorocarbon (e.g., a perfluorocarbon). In one set of embodiments, the fluorinated compound is a polymer (e.g., a fluoropolymer). Alternatively, the fluorinated compound may be non-polymeric (e.g., a non-fluoropolymer). In some embodiments, the fluorinated compound comprises a fluorinated surfactant. Examples of fluorinated compounds may include one or more of polyfluoroalkyl, polyfluoroalkane, perfluoroalkyl, perfluoroalkane, etc. Specific non-limiting examples of fluorinated compounds may include one or more of perfluorodecalin, perflubron, perfluorooctanesulfonic acid, etc.

The fluorinated compounds may be disposed in the in the vessel using any of a variety of appropriate methods and/or carriers described herein. For example, in one set of embodiments, the fluorinated compound may be disposed (e.g., dispersed, suspended) in the vessel in the form of a liquid, a gel, a micelle, a microemulsion, a nanoemulsion, a macroemulsion, a vesicle, and/or a fiber. As described in more detail below, in some embodiments, the fluorinated compounds may be associated with (e.g., contained within) a carrier (e.g., a polymeric or hydrogel scaffold) for deposition in the vessel.

In some embodiments, the oxygen-releasing agent may comprise a material that is incapable of reversibly solubilizing and/or binding oxygen. For example, in one set of embodiments, the oxygen-releasing agent may be capable of undergoing a reaction to produce oxygen. The oxygen-releasing agent may, in some embodiments, react with one or more species (e.g., water molecules, various chemicals, enzymes) contained within the vessel to generate oxygen. The oxygen-releasing agent may be capable of undergoing any of a variety of appropriate reactions, including but not limited to, enzymatic degradation, hydrolytic decomposition, etc. Non-limiting examples of such oxygen-releasing agents includes various types of peroxides.

For example, in some embodiments, the oxygen-releasing agent includes a peroxide such as a solid peroxide (e.g., a solid inorganic peroxide) and/or a liquid peroxide. A non-limiting example of liquid peroxide is hydrogen peroxide. Non-limiting examples of inorganic solid peroxides include one or more of calcium peroxide, magnesium peroxide, sodium percarbonate, etc. The oxygen-releasing agent may be present in any of a variety of appropriate forms, e.g., such as a liquid, a solid, or a gel. In one set of embodiments, the peroxides (e.g., solid peroxides) may be associated with (e.g., contained within) a carrier (e.g., polymeric and/or hydrogel network in the form of a microparticle and/or nanoparticle).

The oxygen-releasing agent may be present within the vessel in any of a variety of appropriate amounts. For example, in one set of embodiments, the oxygen-releasing agent may have a concentration of greater than or equal to 1 mg/mL, greater than or equal to 10 mg/mL, greater than or equal to 20 mg/mL, greater than or equal to 50 mg/mL, greater than or equal to 100 mg/mL, greater than or equal to 250 mg/mL, or greater than or equal to 500 mg/mL. In some embodiments, the oxygen-releasing agent may have a concentration of less than or equal to 1 g/mL, less than or equal to 500 mg/mL, less than or equal to 250 mg/mL, less than or equal to 100 mg/mL, less than or equal to 50 mg/mL, less than or equal to 20 mg/mL, or less than or equal to 10 mg/mL. Combination of the above-reference ranges are possible (e.g., greater than or equal to 1mg/mL and less than or equal to 1 g/mL. or greater than 10 mg/mL and less than or equal to 1 g/mL). Other ranges are also possible.

The oxygen-releasing agent may be disposed within the carrier using any of a variety of appropriate methods described herein. For example, in one set of embodiments, the oxygen-releasing agent may be associated with (e.g., contained within, encapsulated by, linked to, etc.) a carrier having a polymeric and/or hydrogel network or scaffold. As shown in FIG. 1, the carrier 30 may have a polymeric and/or hydrogel network and the oxygen-releasing agent 35 may be contained within the network. The carrier may be present in any appropriate forms such as a microparticle, a nanoparticle, etc. The oxygen-releasing agent may be associated with the polymeric and/or hydrogel network in any appropriate manners, either via chemical associations (e.g., via forming covalent bonds) or physical association (e.g., via forming non-covalent bonds and/or physical entrapment). The carrier may comprise any appropriate types of polymers or hydrogels. In some cases, the carrier comprises a biodegradable polymer.

While FIG. 1 shows a set of embodiments in which the oxygen-releasing agent is disposed within the vessel via a carrier having a polymeric and/or hydrogel network, it should be understood that not all embodiments described herein are so limiting, and in other embodiments, the oxygen-releasing agent may be disposed within the vessel via any appropriate means and/or employing any types of carriers, as described in more detail below.

The oxygen-releasing agent may be disposed within a vessel with or without the use of a carrier. As noted above, in some cases, the oxygen-releasing agent may be associated (e.g., encapsulated within) any of a variety of carriers, including but not limited to, a collapsible bag (e.g., dialysis bag, a tea bag), a droplet or particle (e.g., emulsion droplet, a micelle, a vesicle, a microcarrier, a nanoparticle, a microparticle, a microcapsule), a hydrogel, a gel, a film, a slab, a pill, etc. FIGS. 4A-4D illustrate a non-limiting embodiment of various forms of carriers that may be employed to contain the oxygen-releasing agent. For example, in FIG. 4A, the oxygen-releasing agent 35 may be associated with a microcarrier (e.g., a microparticle) or a nanocarrier (e.g., nanoparticle) 30A. In some cases, as shown in FIG. 4B, the oxygen-releasing agent may be disposed on or within a film or slab 30B. Alternatively or additionally, as shown in FIG. 4C, the oxygen-releasing agent 35 may be disposed as (a part of) a coating 30C on a vessel wall 21. Alternatively or additionally, as shown in FIG. 4D, the oxygen-releasing agent may be disposed or entrapped in a hydrogel scaffold 30D.

Alternatively, in some embodiments, the oxygen-releasing agent may be directly disposed into the vessel without the use of a carrier. For example, the oxygen-releasing agent may be directly disposed into the vessel in the form of a solid (e.g., solid nanoparticle, solid microparticle, a pill, a slab, etc.), a gel, or a liquid. The oxygen-releasing agent may be disposed in any of a variety of locations within the vessel. In some cases, the oxygen-releasing agent, optionally contained within a carrier, may be suspended (e.g., dispersed) in the fluid containing the plurality of cells, e.g., as shown in FIG. 1. In some cases, the oxygen-releasing agent may be immobilized to a portion of the vessel. In one set of embodiments, the oxygen-releasing agent may be immobilized onto an inner portion (e.g., an internal wall) of the vessel that is in fluidic communication with the plurality of cells. For example, as shown in FIG. 1, an internal wall 21 of the vessel 11 may be coated with the oxygen-releasing agent 35. In some embodiments, the interior of the vessel may be coated with a carrier (e.g., a hydrogel and/or polymer matrix) containing the oxygen-releasing agent. In some such cases, the inner wall may be coated with the oxygen-releasing agent during manufacturing of the vessel and/or prior to using the vessel for bioprocessing and/or reactions.

In some embodiments, the carriers described herein may contain one or more magnetic nanoparticles or beads configured to aid the removal of the carrier after bioprocessing. For example, the magnetic particle laden carriers may be sorted and retrieved after each use.

As noted above, in some embodiments, a carrier described herein may be employed as a platform for cell culturing. For example, in one set of embodiments, a microcarrier (e.g., a microparticle, etc.) may be configured to contain an oxygen-releasing agent and at least one cell. For example, the at least one cell may be disposed on or attached to a surface of the microcarrier. The presence of the oxygen-releasing agent in the carrier may advantageously lead to the proliferation of the at least one cell on the carrier. Such a carrier may be employed for cell culture applications, e.g., such as culturing adherent cell lines into a suspension cell culture platform. The use of a carrier (e.g., a microcarrier) comprising an oxygen-releasing agent may allow for higher oxygen delivery to the one or more cells and may potentially improve the cell growth, productivity, and/or other desired functionalities.

A non-limiting representation of a microcarrier containing an oxygen-generating agent is shown in FIG. 5. As shown, a microcarrier 30E may initially contain an oxygen-generating agent 35 and at least one cell 15 seeded onto the microcarrier 30E. The oxygen-generating agent 15 may be configured to release and supply oxygen in-situ to the at least one cell 15 disposed on the microcarrier 30E. Accordingly, the at least one cell may be configured to proliferate on the microcarrier, e.g., as shown by microcarrier 30F.

In some embodiments, a microcarrier described above with respect to FIG. 5 may be advantageously employed in the formation and growth of organoids and spheroids. For example, referring again to FIG. 5, the presence of oxygen-releasing agent 35 within the microcarrier 30 may allow for efficient cell proliferation and formation of an organoid or spheroid.

The microcarrier may have any of a variety of shapes and sizes. In some embodiment, the microcarrier may have a particular size that allows for efficient oxygen diffusion to the at least one cell. In some embodiments, the microcarrier may have a size (e.g., an average cross-sectional dimension (e.g., diameter, width, height, thickness, etc.)) of no more than 1000 micrometers, no more than 500 micrometers, no more than 200 micrometers, no more than 100 micrometers, no more than 50 micrometers, no more than 10 micrometers, no more than 5 micrometers, no more than 1 micrometer, no more than 500 nm, no more than 200 nm, no more 100 nm, no more than 50 nm, or no more than 25 nm. In some embodiments, the microcarrier may have a size range (e.g., an average cross-sectional dimension (e.g., diameter, width, height, thickness, etc.)) of at least 10 nm, at least 25 nm, at least 50 nm, at least 100 nm, at least 200 nm, at least 500 nm, at least 1 micrometer, at least 5 micrometers, at least 10 micrometers, at least 50 micrometers, at least 100 micrometers, at least 200 micrometers, or at least 500 micrometers. Combination of the above-reference ranges are possible (e.g., between 10 nm and 500 micrometers, between 10 nm and 1000 micrometers). Other ranges are also possible.

In some embodiments, the microcarrier may have a relatively short oxygen diffusion distance. For example, as shown in FIG. 5, the oxygen diffusion distance may be the distance released oxygen from the oxygen-releasing agent 35 need to diffuse before reaching the at least one cell 15 within the microcarrier 30E. In some embodiments, the oxygen-diffusion distance within the microcarrier may be no more than 200 micrometers, no more than 100 micrometers, no more than 50 micrometers, no more than 10 micrometers, no more than 5 micrometers, no more than 1 micrometer, no more than 500 nm, no more than 200 nm, no more 100 nm, no more than 50 nm, no more than 25 nm, or no more than 10 nm. In some embodiments, the oxygen-diffusion distance within the microcarrier may at least 5 nm, at least 10 nm, at least 25 nm, at least 50 nm, at least 100 nm, at least 200 nm, at least 500 nm, at least 1 micrometer, at least 5 micrometers, at least 10 micrometers, at least 50 micrometers, or at least 100 micrometers. Combination of the above-reference ranges are possible (e.g., between 5 nm and 200 micrometers). Other ranges are also possible.

The vessel described herein may be configured to contain any of a variety of types of cells. In some embodiments, the cells may include any type of mammalian cells. Non-limiting examples of mammalian cells include Chinese Hamster Ovary (CHO) cells, human embryonic kidney (HEK) cells, stem cells (e.g., mouse embryonic stem (MES) cells, human embryonic stem (HES) cells, induced pluripotent stem (iPS) cells, etc.), T cells, etc.

In some embodiments, the oxygen-releasing agent may be introduced into the vessel at any of a variety of appropriate time intervals and/or frequencies throughout the bioprocessing and/or biochemical reaction. For example, in some cases, as the number of cells within the vessel exceeds a threshold value and/or when the amount of dissolved oxygen is lower than a threshold value, the amount of oxygen level within the vessel may be incapable of sustaining the cells. Accordingly, it may be advantageous to repeatedly introduce an oxygen-releasing agent into the vessel to promote cell viability. The oxygen-releasing agent may be introduced into the vessel using any appropriate methods. For example, in one set of embodiments, the system may comprise a pump configured to introduce the oxygen-releasing agent into the vessel with an inlet opening or port.

In some embodiments, the oxygen-releasing agent may generate any appropriate amount of oxygen per volume of oxygen-releasing agent. In some embodiments, the oxygen-releasing agent may be capable of releasing (or generating) greater than or equal to 10 mg/L, greater than or equal to 15 mg/L, greater than or equal to 20 mg/L, greater than or equal to 25 mg/L, greater than or equal to 30 mg/L, greater than or equal to 35 mg/L, greater than or equal to 40 mg/L, greater than or equal to 45 mg/L, greater than or equal to 50 mg/L, or greater than or equal to 55 mg/L of amount of oxygen per volume of the oxygen-releasing agent. In some embodiments, the oxygen-releasing agent may be capable of releasing (or generating) less than or equal to 60 mg/L, less than or equal to 55 mg/L, less than or equal to 50 mg/L, less than or equal to 45 mg/L, less than or equal to 40 mg/L, less than or equal to 35 mg/L, less than or equal to 30 mg/L, less than or equal to 25 mg/L, less than or equal to 20 mg/L, or less than or equal to 15 mg/L of amount oxygen per volume of the oxygen-releasing agent. Combination of the above-reference ranges are possible (e.g., greater than or equal to 10 mg/L and less than or equal to 60 mg/L). Other ranges are also possible.

The vessel may comprise a plurality of cells having any of a variety of cell densities. In some cases, the presence of the oxygen-releasing agent may advantageously sustain a relatively high cell density within the vessel. In some embodiments, the vessel comprises cells having a cell density of greater than or equal to 5 million cells per mL, greater than or equal to 10 million cells per mL, greater than or equal to 20 million cells per mL, greater than or equal to 40 million cells per mL, greater than or equal to 50 million cells per mL, greater than or equal to 60 million cells per mL, greater than or equal to 80 million cells per mL, greater than or equal to 100 million cells per mL, greater than or equal to 200 million cells per mL, greater than or equal to 300 million cells per mL, or greater than or equal to 400 million cells per mL. In some embodiments, the vessel comprises cells having a cell density of less than or equal to 500 million cells per mL, less than or equal to 400 million cells per mL, less than or equal to 300 million cells per mL, less than or equal to 200 million cells per mL, less than or equal to 100 million cells per mL, less than or equal to 80 million cells per mL, less than or less than or equal to 60 million cells per mL, less than or equal to 40 million cells per mL, less than or equal to 20 million cells per mL, or less than or equal to 10 million cells per mL. Combination of the above-reference ranges are possible (e.g., greater than or equal to 5 million cells per mL and less than or equal to 500 million cells per mL). Other ranges are also possible.

As noted above, the system may further comprise an ancillary agent disposed in the vessel. The ancillary agent may advantageously assist with the removal of gaseous byproducts from the system. For example, the ancillary agent may have an affinity for a gaseous product produced from a bioprocessing and/or biochemical process associated with the plurality of cells. In some cases, the ancillary agent may associate (e.g., bind to, absorb, interact, etc.) with a gaseous product comprising CO₂. In some embodiments, the ancillary agents comprises one or more of amines, hydroxides, minerals, zeolites. In some cases, the ancillary agents comprises an alkaline solution. Specific non-limiting examples of ancillary agents includes monoethanolamine, lithium hydroxide, sodium hydroxide, etc.

Alternatively or additionally, the ancillary agents comprises a material capable of reacting with and/or utilizing the gaseous product (e.g., CO₂). Non-limiting examples include photosynthetic materials comprising biomass and/or photosynthetic cells.

Alternatively or additionally, the ancillary agent comprises an electrochemical component (e.g., electrochemical cell) capable of electrochemically capturing or converting a gaseous product (e.g., CO₂) from a fluid. For example, the electrochemical component may comprise a material capable of binding the gaseous species during an electrochemical process. In some cases, the material may be coated onto one or more electrodes and may bind to the gaseous product during charging of the electrochemical device. Non-limiting examples of such a material may include a quinone comprising polyanthraquinone, carbon nanotubes, amines (e.g., monoethanolamine), hydroxides (e.g., sodium hydroxide, lithium hydroxide, etc.), minerals, zeolites, etc.

The ancillary agent described herein may be disposed in the vessel in any of a variety of configurations. In some embodiments, the ancillary agent may be disposed in the vessel in a substantially similar manner as the oxygen-releasing agent. For example, the ancillary agent may be associated with (e.g., contained within) any of a variety of carriers (e.g., microparticle, microcapsule, hydrogel and/or polymeric matrix, tea bag, dialysis bag, etc.) described herein or with respect to FIGS. 1-2. Alternatively, the ancillary agent may be directly disposed (e.g., dispersed, suspended, etc.) in a fluid within the vessel, or coated onto an interior portion of the vessel. The ancillary agent may be present in any appropriate forms, including but not limited to a solid, liquid, gel, etc.

In some embodiments, the vessel may be configured to contain reactants, media, and/or other components necessary for carrying out a desired process such as a chemical, biochemical and/or biological reaction. The vessel may be a container having any of a variety of appropriate properties. Non-limiting examples of a vessel may include a collapsible container (i.e., a container without a self-supporting structure), a container having a rigid and re-usable support structure (e.g., a stainless-steel tank), or a semi-rigid support structure (e.g., a polymeric container). Additionally and/or alternatively, all or portions of the container may comprise a substantially rigid material such as a rigid polymer, metal, and/or glass. The vessel may also any of a variety of sizes and volumes, e.g., such as formed from a microfluidic or millifluidic channel, as described elsewhere herein.

In some embodiments, as described above, the vessel may be a part of a millifluidic or microfluidic system and may have a relatively small size and volume as described elsewhere herein. However, it should be noted that the vessel may have any of a variety of appropriate sizes. For example, in some embodiments, the vessel may have a volume of greater than or equal to 1 μL, greater than or equal to 10 μL, greater than or equal to 100 μL, greater than or equal to 1 mL, greater than or equal to 5 mL, greater than or equal to 15 mL, greater than or equal to 25 mL, greater than or equal to 50 mL, greater than or equal to 100 mL, greater than or equal to 150 mL, greater than or equal to 250 mL, greater than or equal to 500 mL, greater than or equal to 1 L, or greater than or equal to 1.5 L. In some embodiments, the vessel may have a volume of less than or equal to 2 L, less than or equal to 1.5 L, less than or equal to 1 L, less than or equal to 500 mL, less than or equal to 250 mL, less than or equal to 150 mL, less than or equal to 100 mL, less than or equal to 50 mL, less than or equal to 25 mL, less than or equal to 15 mL, less than or equal to 5 mL, less than or equal to 1 mL, less than or equal to 100 μL, or less than or equal to 10 μL. Combination of the above-reference ranges are possible (e.g., greater than or equal to 15 mL and less than or equal to 250 mL, greater than or equal to 15 mL and less than or equal to 2 L, or greater than or equal to 1 μL and less than or equal to 2 L). Other ranges are also possible.

As shown in FIGS. 1-2, the vessel may comprise one or more optional openings configured to facilitate more convenient introduction and removal of a substance and/or gas from the container. The vessel may have any suitable number of inlet ports and any suitable number of outlet ports. For example, a plurality of inlet ports may be used to provide different gas compositions (e.g., via a plurality of conduit 125 in FIG. 2), and/or to allow separation of gases prior to their introduction into the vessel. These openings or ports may be positioned in any suitable location with respect to vessel. For instance, for some vessels including spargers, the vessel may include one more gas inlet ports located at a bottom portion of the vessel. Tubing may be connected to the inlet and/or outlet ports to form, e.g., delivery and harvest lines, respectively, for introducing and removing liquid from the vessel.

Optionally, the vessel and/or support structure may include a utility tower, which may be provided to facilitate interconnection of one or more devices internal to the vessel and/or support structure with one or more pumps, controllers, and/or electronics (e.g., sensor electronics, electronic interfaces, and pressurized gas controllers) or other devices. Such devices may be controlled using a control system, as described in more detail below.

As described above, the vessel may optionally include a mixing mechanism (e.g., an impeller, an agitator, etc.). The mixing mechanism described herein may advantageously allow the system to mix fluids, solids, or foams of any type. For example, fluids inside the container may be mixed to provide distribution of various components (e.g., cells, nutrients, oxygen-releasing agents, ancillary agents) and dissolved gases for cell growth applications.

In some cases, more than one agitator or mixer may be used, and the agitators and/or mixes may the same or different. More than one agitation system may be used, for example, to increase mixing power. In some cases, the agitator may be one in which the height can be adjusted, e.g., such that the draft shaft allows raising of an impeller or agitator above the bottom of the tank and/or allows for multiple impellers or agitators to be used.

Although many of the figures described herein (e.g., FIG. 2) shows an impeller that is positioned at or near a bottom portion of a container, in other embodiments, impellers can be positioned at any suitable location within a container, for example, near the center or a top portion of a container. This can be achieved by extending the length of a shaft which supports the impeller, or by any other suitable configuration. Positions of impellers in a container may depend on the process to be performed in the container. For instance, in some embodiments where sparging is present, impellers may be positioned near the sparger such that the impeller can sweep and/or regulate the gas bubbles (e.g., oxygen gas, etc.) introduced into the vessel. Additionally, although the figures described herein show a single impeller associated with a shaft, more than one impeller can be used in some instances. The first impeller may provide adequate sweeping of a gas (e.g., dissolved oxygen), and the second impeller may provide adequate mixing of contents within the container.

In some embodiments, the vessel may optionally include a heating system (e.g., such as an internal or external heating mechanism). Any of a variety of appropriate heating systems may be employed to heat the substances within the vessel to a temperature necessary for carrying out bioprocessing and/or a biochemical reaction.

As noted above, a vessel, such as vessels 11 and 111 shown in FIGS. 1-2, may include various sensors and/or probes for controlling and/or monitoring one or more process parameters inside the vessel such as, for example, temperature, pressure, pH, dissolved oxygen (DO), dissolved carbon dioxide (DCO₂), mixing rate, and gas flow rate. The sensor may also be an optical sensor in some cases.

In some cases, sensors and/or probes (e.g., sensor 118 in FIG. 2) may be connected to a sensor electronics module, the output of which can be sent to a terminal board and/or a relay box. The results of the sensing operations may be input into a computer-implemented control system (e.g., a computer) for calculation and control of various parameters (e.g., amount of dissolved oxygen in the fluid containing the plurality of cells). Such a control system may also include a combination of electronic, mechanical, and/or pneumatic systems to control heat, oxygen, and/or liquid delivered to or withdrawn from the disposable container as required to stabilize or control the environmental parameters of the process operation. It should be appreciated that the control system may perform other functions and the invention is not limited to having any particular function or set of functions.

In one set of embodiments, the control system is a closed-loop controller. In some cases, the control system, upon receiving an input of a measured property (e.g., dissolved oxygen level, carbon dioxide level, etc.) from a sensor positioned in the vessel, may in turn trigger the vessel to control the property within vessel. For example, in one set of embodiments, upon sensing a dissolved oxygen level below a desired threshold level within the vessel, the control system may be capable of triggering the addition of oxygen-releasing agents and/or oxygen gas into the vessel. As another example, in one set of embodiments, upon sensing a carbon dioxide level above a desired threshold level within the vessel, the control system may be capable of triggering the addition of one or more ancillary agents into the vessel to remove excess carbon dioxide.

The one or more control systems can be implemented in numerous ways, such as with dedicated hardware and/or firmware, using a processor that is programmed using microcode or software to perform the functions recited above or any suitable combination of the foregoing. A control system may control one or more operations of a single reactor for a biological, biochemical or chemical reaction, or of multiple (separate or interconnected) reactors.

Each of systems described herein (e.g., with reference to FIGS. 1-2), and components thereof, may be implemented using any of a variety of technologies, including software (e.g., C, C#, C++, Java, or a combination thereof), hardware (e.g., one or more application-specific integrated circuits), firmware (e.g., electrically-programmed memory) or any combination thereof.

Various embodiments according to the invention may be implemented on one or more computer systems. These computer systems, may be, for example, general-purpose computers such as those based on Intel PENTIUM-type and XScale-type processors, Motorola PowerPC, Motorola DragonBall, IBM HPC, Sun UltraSPARC, Hewlett-Packard PA-RISC processors, any of a variety of processors available from Advanced Micro Devices (AMD) or any other type of processor. It should be appreciated that one or more of any type of computer system may be used to implement various embodiments of the invention. The computer system may include specially-programmed, special-purpose hardware, for example, an application-specific integrated circuit (ASIC). Aspects of the invention may be implemented in software, hardware or firmware, or any combination thereof. Further, such methods, acts, systems, system elements and components thereof may be implemented as part of the computer system described above or as an independent component.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to

“A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A system for bioprocessing, comprising:
a vessel comprising an inlet configured to introduce substances into the vessel, wherein the vessel comprises at least one microfluidic or millifluidic channel;
a plurality of cells contained within the vessel; and
an oxygen-releasing agent disposed within the vessel.

2. A system for bioprocessing, comprising:
a vessel comprising an inlet configured to introduce substances into the vessel;
a plurality of cells suspended in a fluid or attached to a carrier contained within the vessel; and
an oxygen-releasing agent disposed within the vessel, wherein the oxygen-releasing agent is incapable of reversibly solubilizing and/or binding oxygen.

3. A method, comprising:
providing a system comprising a vessel, wherein the vessel comprises an inlet and at least one microfluidic or millifluidic channel;
disposing an oxygen-releasing agent into the vessel;
introducing at least one cell into the vessel; and
releasing oxygen in the vessel from the oxygen-releasing agent.

4. The method of claim 3,
wherein the oxygen-releasing agent is incapable of reversibly solubilizing and/or binding oxygen;

5-15. (canceled)

16. The system of claim 1, wherein the oxygen-releasing agent is encapsulated within a dialysis bag and/or a tea bag.

17-19. (canceled)

20. The system of claim 1, wherein the oxygen-releasing agent is associated with a magnetic agent contained within a carrier.

21-26. (canceled)

27. The system of claim 1, wherein the oxygen-releasing agent comprises a fluorinated compound comprising a fluorocarbon.

28. The system of claim 27, wherein the fluorinated compound is non-polymeric.

29. The system of claim 27, wherein the fluorinated compound comprises on or more of a perfluorinated compound, a fluoropolymer, and a fluorinated surfactant.

30-31. (canceled)

32. The system of claim 1, wherein the oxygen-releasing agent comprises a fluorinated compound comprising one or more of perfluorodecalin, perflubron, perfluorooctanoic acid, and perfluorooctanesulfonic acid.

33. The system of claim 1, wherein the oxygen-releasing agent comprises a fluorinated compound dispersed in the vessel in the form of a gel, a micelle, a microemulsion, a nanoemulsion, a macroemulsion, a vesicle, and/or a fiber.

34. The system of claim 1, further comprising an ancillary agent disposed within the vessel having an affinity for a gaseous product, and wherein the gaseous product comprises CO₂.

35. The system of claim 34, wherein the ancillary agent comprises an amine, a hydroxide, a mineral, and/or a zeolite.

36. The system of claim 34, wherein the ancillary agent comprises biomass and/or photosynthetic cells.

37. The system of claim 34, wherein the ancillary agent comprises an electrochemical cell.

38. (canceled)

39. The system of claim 1, wherein the system further comprises a conduit in fluid communication with the vessel configured to introduce a gas into the vessel.

40. They system of claim 39, wherein the conduit comprises a sparger.

41-45. (canceled)

46. The system of claim 1, wherein the vessel further comprises a mixing mechanism disposed within the vessel.

47. The system of claim 1, wherein the vessel further comprises a pump configured to introduce the oxygen-releasing agent via the inlet.

48. The system of claim 1, wherein the system further comprises a heating system.

49-55. (canceled)
